(19) United States
(12) Patent Application Publication
(10) Pub. No.: US 2023/0190351 A1
(43) Pub. Date: Jun. 22, 2023

(54) JOINT FUSION SYSTEMS AND DEVICES

(71) Applicant: IN-SITU ORTHOPEDICS, LLC, Houston, TX (US)

(72) Inventors: JOSEPH JOHNSTON, Houston, TX (US); PEDRO COSCULLUELA, Houston, TX (US); TRAVIS BRYAN, Houston, TX (US); THOMAS BLACKBURN, Houston, TX (US)

(73) Assignee: IN-SITU ORTHOPEDICS, LLC, Houston, TX (US)

(21) Appl. No.: 18/068,602

(22) Filed: Dec. 20, 2022

Related U.S. Application Data
(60) Provisional application No. 63/292,118, filed on Dec. 21, 2021.

(51) Int. Cl. A61B 17/88; A61B 17/80; A61F 2/46

(52) U.S. Cl. CPC A61B 17/8863; A61B 17/8061; A61F 2/4606; A61B 2017/565

(57) ABSTRACT

A device for bending joint fusion plates to a precise degree of bend, and methods of using same. The bender has three pressure points, two that sit under the plate and one above, and compressing the handle will bend the plate to a desired degree as there is a stop point that can be set by the user for the degree of bend. The bender allows the surgeon to bend the plate prior to surgery, thus avoiding having to bend the plate during the surgery as in current practice. Kits and systems are also provided.

### PRIOR RELATED APPLICATIONS

This application claims priority to 63/292,118, filed Dec. 21, 2021 and is incorporated by reference in its entirety for all purposes.

### FEDERALLY SPONSORED RESEARCH STATEMENT

Not applicable.

### FIELD OF THE DISCLOSURE

The disclosure generally relates to systems, devices, and methods for joint fusions, such as hallux metatarsophalangeal fusions.

### BACKGROUND OF THE DISCLOSURE

The first metatarsophalangeal joint, also called the first MTP or hallux MTP, is the big toe joint (see FIG. 1). It is a cam shaped, hinged joint providing a normal alignment of the big toe from 5° valgus (toe tipping outward from centerline of body) to 15 valgus, and normally allowing 70-90° of dorsiflexion (upwards) and 35-50° plantarflexion (downwards).

When the cartilage in the MTP joint wears away, the two bones that make up the big toe joint rub against one another. This can cause severe pain and difficulty in both wearing shoes and in walking. Arthrodesis or “fusion” of the MTP joint is a common surgery that is typically performed for the treatment of hallux rigidus— arthritis of the hallux MTP joint—and for some cases of hallux valgus, also known as a bunion.

In a MTP joint fusion, the bones are joined (fused or welded) together permanently so they cannot rub against each other and cause further pain. The surgery is performed as an outpatient procedure under general anesthesia. After the surgery, a local numbing medicine similar to Novocain is placed in the foot to keep patients comfortable. The surgery begins with a small incision over the big toe joint. The cartilage within the joint is removed with special instruments called burrs or reamers. These instruments make fitting the bones back together in perfect position easier.

The joint is then placed in the precisely correct position and stabilized with small screws and in many cases a small titanium plate is also used to stabilize the bones. These screws and/or plates are usually permanent and are not removed. For example, FIG. 2 shows a rigid plate 200 attached to the phalanx 202 and metatarsal 204 via screws 206, preventing movement of the MTP joint 208. The most common surgery uses a locking plate and a lag screw, providing optimal stability, but many variations of the MTP joint fusion are possible.

The procedure carries a success rate of about 85% in the literature, and one of the most common reasons for dissatisfaction is the position of the big toe after the fusion is achieved. The goal for the position of the toe is in a slight amount of dorsiflexion that can accommodate a ¾-to 1 inch heel. If the toe is fused too low, there is pressure with weightbearing under the hallux interphalangeal (IP) joint and the tip of the toe. If the toe is fused too high, the top of the toe will rub against the shoe causing pain. In addition, a deformity of the hallux IP may develop as the patient tries to place the toe on the ground when standing.

There are many patents directed to MTP joint fusion plates, and most are rigid structures with holes for the screws and available in 0°, 5° and 10° dorsiflexion angles. U.S. Pat. No. 8,167,918, for example, describes a rigid plate that is curved to fit over a bone and having 6 attachment holes. The plate is pre-contoured so as to fit most applications, but can also be further bent by the surgeon as desirable to suit individual variations in anatomy or circumstance. Achieving the correct bend mid surgery is not trivial, however, and is the cause of many failed fusions and/or dissatisfied patients. Further, repeated attempts to achieve the correct angle will weaken the plate at the bend location, causing premature failure.

Thus, what is needed in the art are better methods and tools for achieving MTP and other joint fusions with accurate and reproduceable bend angles. The ideal methods and devices will allow all of the decision making with regards to the fusion to be made prior to making the first incision on the patient in the operative room.

### SUMMARY OF THE DISCLOSURE

Orthopedic surgery has several procedures for which templating—preoperative planning of implant surgery—is available. These include shoulder, knee and hip replacement surgery. Prior to the procedure, the surgeon looks over the radiographs and uses commercially available software in order to determine the proper fit of the implants. However, there is currently no templating available when it comes to fusion of the hallux metatarsophalangeal joint. We believe—and have now demonstrated—that, unlike some of the other procedures in foot and ankle, the fusion of the hallux MTP joint does lend itself to preoperative planning in order to minimize position-associated issues intra- and post-operatively and this invention addresses that need, amongst others.

Detailed planning for the surgery occurs during the preoperative assessment. A lateral view radiograph of the foot will be taken using a foot board that holds the foot in a fixed position and incorporates an e.g., ¾ inch heel with the toe planted on the foot board. The resulting radiograph allows the surgeon to accurately measure the angle created when a line is drawn along the top of the hallux metatarsal and the top of the hallux proximal phalanx—the dorsiflexion angle 110. This angle varies from person to person and is determined by the structure and size of the foot. See FIG. 1A-B.

The dorsiflexion angle determination is made using the goniometer feature that is available in most, if not all, of the commercially available PACS software packages. Under the current software, the surgeon places dots to delineate the straight portions of the phalanx and the metatarsal (the bone ends are larger and omitted) and the software connects the dots and measures the angle between the two straight lines. FIG. 1C.

Although the dots are currently placed manually, using a cursor positioned by the surgeon, we anticipate that with AI training against a number of X-rays, the software will eventually be able to provide this function as well.

The plate angle is then changed with the bender described herein to the exact angle specified, and the device autoclaved for surgical use. Alternatively, the components can be presterilized and the bend made in a sterile manner. The accuracy of the angle is confirmed before proceeding, and adjustments made if needed. However, in our experience, the plate bender allows for very accurate bending and very little, if any, adjustment is needed, thus preserving the strength of the plate.

Plate bending is achieved by placing the plate, concave side down in the bender (described in more detail below), which has been pre-dialed for the correct bend angle using the dial at the base of the bender. Ideally, the bender is shaped so as to accept the plate correctly in only a single orientation, but this may be optional where a marking is provided on the plate for bender placement.

In some instances, it may be necessary to calibrate the instrument before use, in which case a set of guides may be provided marked with specific angles. The guide—a metal or very hard plastic strip bent to a known angle—can be placed in the bender and used to confirm the dialed-in angle is correct.

The actual surgery proceeds pretty much as currently practiced, the proviso being that the plate was pre-bent (using the device described herein) to the unique angle required for a given patient during the preoperative procedures, and thus is not bent during surgery.

Alternatively, we have devised a plate-first method to the fusion procedure. In this instance, a plate that is bent according to the pre-operative planning is positioned over the joint after the joint has been prepared. The plate is then fixed in place with olive-wires, which are commercially available, holding the toe in the proper dorsiflexion angle. The plate also has a valgus angle of 10 degrees, ensuring proper varus and valgus alignment to the toe. With the plate holding the toe in proper posture, the trans-articular lag screw hole is then drilled, and the lag screw inserted, compressing the joint. The procedure is then completed by drilling the holes in the plate and attaching it to bone with screws.

There are many plate alternatives in the market today to plate a hallux MTP joint for fusion. However, there are no devices currently available for revision of failed fusions or failed replacements. In order to satisfy these needs, additional plates can be made available that still are mated to fit the bender, yet have different peri-articular screw configurations within the plate boundary. The new screw holes allow for fixation in fresh bone, avoiding previously used screw paths, which can contribute to further failures. Also, the distance between the proximal and distal peri-articular plates can be increased in order to allow for bone block wedges to bridge the gap over any large bony defects.

The invention includes any one or more of the following embodiments, in any combination(s) thereof:

A bender for precise bending of a separate joint fusion plate, said bender comprising:

a) a main body having a hollow therein and having first and second pressure points protruding from an exterior surface near a top end of said main body and a first slot between said first and second pressure points;

b) a stop shaft inside said hollow and means for controlling a height of said stop shaft inside said hollow;

c) a travelling jaw having a lower end at least partially inside said hollow, said travelling jaw having a third pressure point at an upper end of said travelling jaw, said third pressure point positioned between said first and second pressure points in said slot;

d) said travelling jaw able to travel up to allow placement of a separate joint fusion plate between said first and second pressure points and said third pressure point and able to travel down to meet said stop shaft;

e) a handle operatively connected to said main body and to said travelling jaw, said handle for raising and lowering said travelling jaw and for applying a pressure to said third pressure point when said separate joint fusion plate is positioned in a stable and unmoving position supported by said first and second pressure points, thereby bending said separate joint fusion plate.

Any bender herein described, wherein said means for controlling said height of said stop shaft is a rotating dial at a bottom of said main body that raises and lowers said stop shaft via a threaded region that mates with a threaded region inside said hollow. Preferably, the dial has markings thereon for indicating a degree of bending.

Any bender herein described, wherein said travelling jaw is biased to return to a lowered position or wherein said travelling jaw is spring biased to return to a lowered position with a spring connected to said main body and to said travelling jaw.

Any bender herein described, wherein said handle is hingedly connected to said main body and to said travelling jaw via a drag link, said main body having a second slot opposite said first slot, said second slot allowing said drag link to travel up and down said main body with said handle, or wherein said handle is hingedly connected to said main body and to said travelling jaw via a drag link, said main body having a second slot opposite said first slot, said second slot allowing said travelling jaw to travel up and down with said handle, wherein said travelling jaw is spring biased to return to a lowered position, said spring connected to said main body and to said travelling jaw.

Any bender herein described, wherein said device comprises metal or wherein said main body, said handle, and said travelling jaw are made of metal.

Any bender herein described, wherein said first and second pressure points are integral to said main body and wherein said third pressure point is integral to said travelling jaw, or wherein said first and second pressure points are not integral to said main body, but comprise a first separate attachable component, and wherein said third pressure point is not integral to said travelling jaw but comprises a second separate attachable component.

Any bender herein described, wherein said first and second pressure points have an upper surface that mirrors a lower surface of said separate joint fusion plate and said third pressure point has a lower surface that mirrors an upper surface of said separate joint fusion plate.

A system for joint fusions, said system comprising: a metal joint fusion plate having a shape designed to fit against two bones to be fused and a plurality of holes for attachment means to travel therethrough and into said two bones; and any bender herein described. The system may also include a series of known angle bent plates for calibrating said bender.

Any kit for joint fusions, said kit being a container comprising: any bender herein described; and a plurality of metal joint fusion plates having a shape, said shape designed to i) fit against two bones to be fused and a plurality of holes for attachment means to travel therethrough and into said two bones and ii) fit into said bender. Alternatively, the kit may include the herein described bender; a plurality of metal joint fusion plates having a shape, said shape designed to fit against two bones to be fused and a plurality of holes for attachment means to travel therethrough and into said two bones; plus a plurality of first and second separate attachable components (having the requisite first, second and third pressure points) shaped to fit said plurality of metal joint fusion plates and reversibly attach to said bender.

Any kit herein described may further comprise instructions for use, and/or said plurality of metal joint fusion plates being in a plurality of sizes, and/or said plurality of metal joint fusion plates being in a small, medium and large sizes, and/or said kit further comprising at least two known angle bent plates for calibrating said bender. In preferred cases, said shape fitting against a phalanx bone and a metatarsal bone.

A system for metatarsophalangeal (MTP) joint fusions, said system comprising: a MTP joint fusion plate having a shape designed to fit against a phalanx bone and a metatarsal bone and having a plurality of holes for screws to travel therethrough and into said phalanx bone and said metatarsal bone; and any bender herein described.

A method for fusing two bones, said method comprising:

a) obtaining a radiograph of two bones to be fused, said two bones positioned to have a desired angle therebetween;

b) measuring said desired angle from said radiograph;

c) bending a joint fusion plate to have said desired angle using any bender herein described; and

d) surgically installing said bent joint fusion plate onto said two bones, thereby fusing said two bones at said desired angle.

Any method herein described, wherein said bent joint fusion plate is sterilized after bending and before use, or wherein said bent joint fusion plate is sterilized before bending and said bending step c) is performed in a sterile manner.

A method for fusing a phalanx bone and a metatarsal bone, said method comprising:

a) obtaining a radiographic image of a foot having a phalanx bone and a metatarsal bone to be fused by positioning said foot on a ¾-1 inch heel block, applying weight to said foot and taking said radiographic image;

b) measuring an angle between said phalanx bone and said metatarsal bone from said radiographic image;

c) bending a joint fusion plate to have said angle using any bender herein described; and

d) surgically installing said bent joint fusion plate onto said phalanx bone and said metatarsal bone.

These method steps may occur in any suitable order, but in some embodiments, step a-c) may occur before step d) is initiated or steps a-b) occur before step d) is initiated.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim. The phrase “consisting of” is closed, and excludes all additional elements. The phrase “consisting essentially of” excludes additional material elements, but allows the inclusions of non-material elements that do not substantially change the nature of the invention, such as instructions for use, sterile packaging, carrying case, sterilization solutions, and the like. Any claim or claim element introduced with the open transition term “comprising,” may also be narrowed to use the phrases “consisting essentially of” or “consisting of,” and vice versa. However, the entirety of claim language is not repeated verbatim in the interest of brevity herein.

The following abbreviations are used herein:

| ABBREVIATION | TERM |
|---|---|
| MTP | Metatarsophalangeal |
| IP | Interphalangeal |

### BRIEF DESCRIPTION OF THE DRAWINGS

FIG. 1A Side view of foot with raised heel showing phalanx 102, metatarsal 104 and MTP joint 108.

FIG. 1B Top view of foot showing the hallux vargus angle 112 and the intermetatarsal angle 114.

FIG. 1C Lateral radiograph of a patient recommended for an MTP fusion, showing dots indicating straight portions of each bone, lines connecting each and the dorsiflexion angle 110 therebetween.

FIG. 2 Perspective view of foot with shows rigid plate 200 attached to the phalanx 202 and metatarsal 204 via screws 206, preventing movement of the MTP joint 208.

FIG. 3A-E shows various embodiments or views of a toe plate. FIG. 3A is a top view. FIG. 3B is a cross section at line B-B and FIG. 3C is a cross section at line C-C. FIG. 3D is a variant design (top view) for the toe plate, and here we see right and left toe plates that are mirror images of each other. FIG. 3E shows two plates with differing screw positions—one from 3D and another with shifted screw positions, allowing use of the second plate where the first fusion has failed. The third panel shows the two overlaid.

FIG. 4A-L shows various views of the bender. FIG. 4A is a front view (handle in back) with a simplified rectangular plate shown in position and FIG. 4B a side view. FIG. 4C is a top view of the bender. FIG. 4D is a cross section view from the side, and FIG. 4E-F are enlargements. FIG. 4G is the travelling jaw alone, and FIG. 4H is the drag link alone. FIG. 4I is the angle set shaft alone, and FIG. 4J is the calibrator and stop shaft alone. FIG. 4K is another view of the dial, and FIG. 4L is a cross section of the posts showing the curved upper surface that matches the curve of the toe plate in FIG. 3C.

FIG. 5A-D shows an alternate embodiment with detachable posts and bender point, thus allowing customization of the tool for each type of plate. FIG. 5A compares the original and new main body and travelling jaw with the detachable posts. FIG. 5B shows the components assembled with screws. FIG. 5C compares the original and new travelling jaw with detachable bender point, and FIG. 5D shows them assembled. FIG. 5E shows the original embodiment of FIG. 4 and new embodiment of FIG. 5 side by side.

FIG. 6 shows an alternate embodiment of a toe plate with guide bumps, ensuring that the bender pressure points sit in the required position on the toe plate.

FIG. 7 shows the combined toe plate of FIG. 6 together with the alternate embodiment of the tool of FIG. 5.

### DETAILED DESCRIPTION

The disclosure provides novel joint fusion plates, as well as a dedicated bender device that allows a very precise and reproducible degree of bending of any plate. We exemplify the invention herein with respect to a hallux MTP plate, but the same principles can be applied to any joint fusion plate, and the shapes adjusted accordingly.

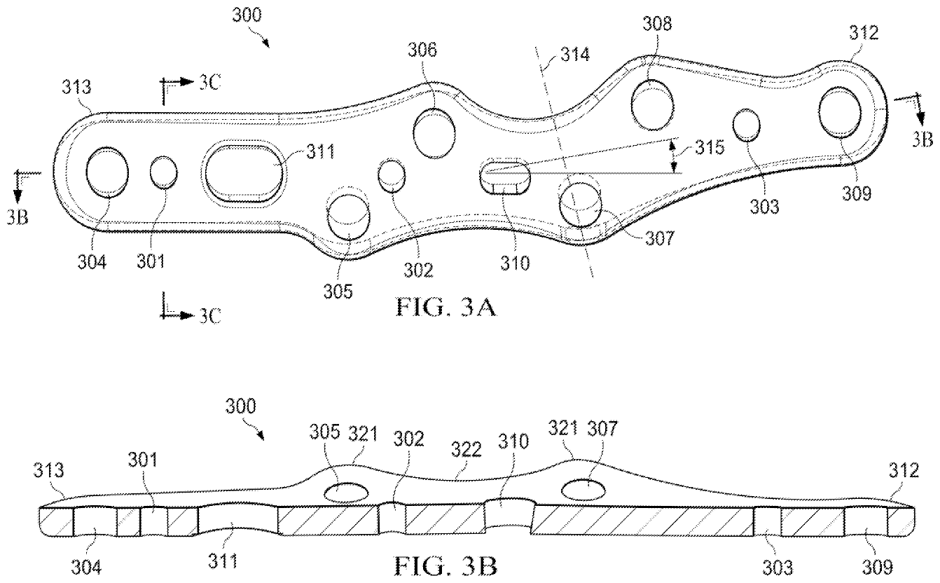

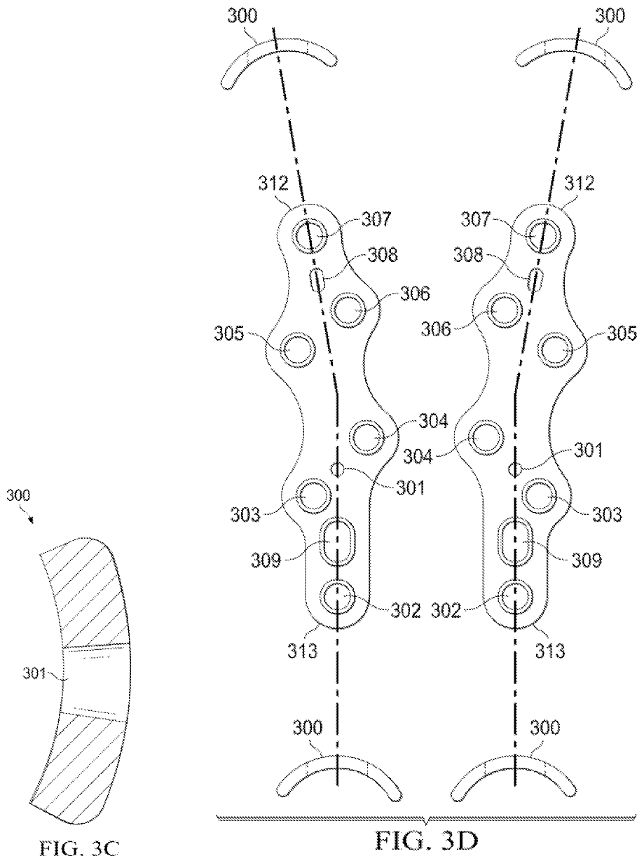

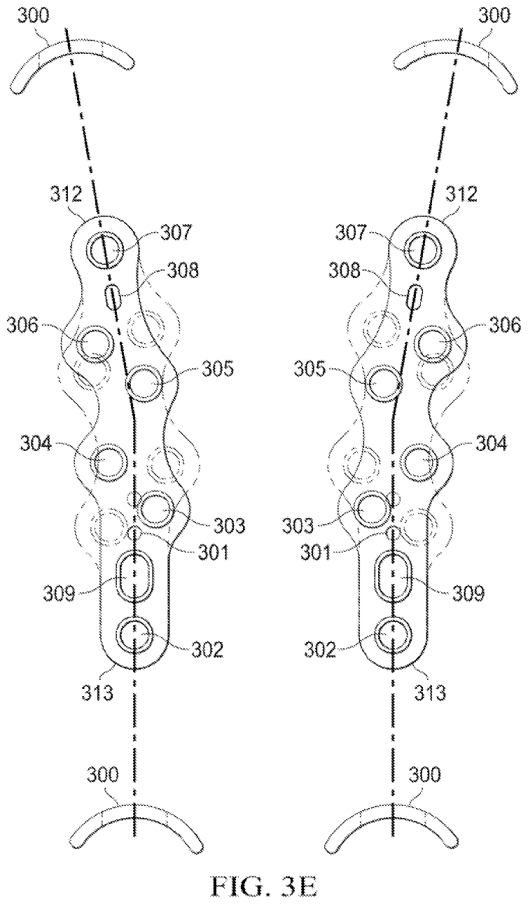

FIG. 3A-C shows one exemplary joint fusion plate. The overall shape of the plate can vary as needed for different joints, but here in the top view of FIG. 3A we see a hallux plate 300 with a generally linear shape, but with a slight bend (see angle 315) to accommodate the normal valgus inclination of the big toe. Also seen are 4 protrusions or bumps 321 on the perimeter, allowing screw holes to be placed near the outer edges of the toe bones, the 4 bumps bracket a narrowed region 322 (corset shaped or bi-convex) that overlies the interface of the two bones. Distal (toe) end of the plate is shown at 312, and proximal end at 313.

FIG. 3B is a cross section at line B-B and bumps 321 that brackets waist 322 are also labelled. FIG. 3C is a cross section at line C-C where the arcuate cross section that will accommodate a generally cylindrical bone can be seen.

In more detail, FIG. 3A is a top view of the toe plate 300. Screw holes 304, 305, 306, 307, 308, and 309 penetrate the plate 300 and provide a place for fasteners to attach the plate to the two bones. Larger hole 311 is also a screw hole but allows for back and forth adjustment of the plate along the bones. Hole 310 allows for medical imaging of the joint to ensure that the two surfaces are held in correct and tight juxtaposition. Smaller holes 301, 302 and 303 allow for K-wires to be used. The placement of these various holes may vary. Bend line 314 is shown as a dotted line and indicates where the plate will be bent.

The fasteners themselves are not shown, and can be any known in the art or to be developed, including locking screws, such as those by Flower Orthopedics, or self-compressing screws that have an asymmetrical head and function to pull the joints together as they are tightened and seat in a specially shaped screw head in the plate, such as the general purpose compression bone screw by TST Medical Devices.

FIG. 3D is another shape for the toe plate, and FIG. 3E shows a secondary use plate overlaid with the plate of FIG. 3D indicating the shifted hole positions. The parts are otherwise the same. Since the holes are shifted, this plate can be used in a secondary joint fusion operation when the first fails.

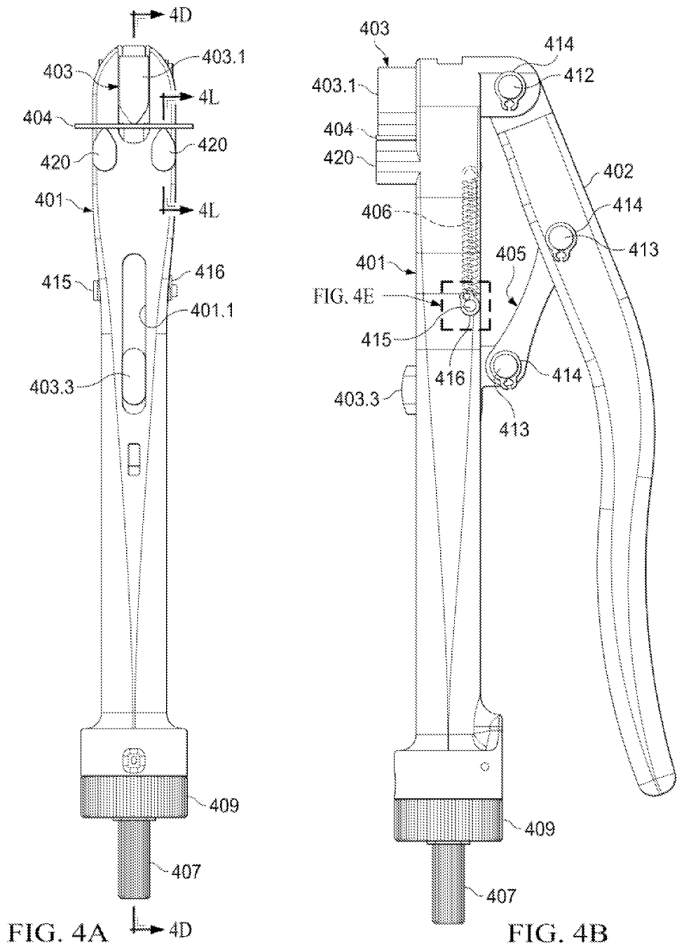

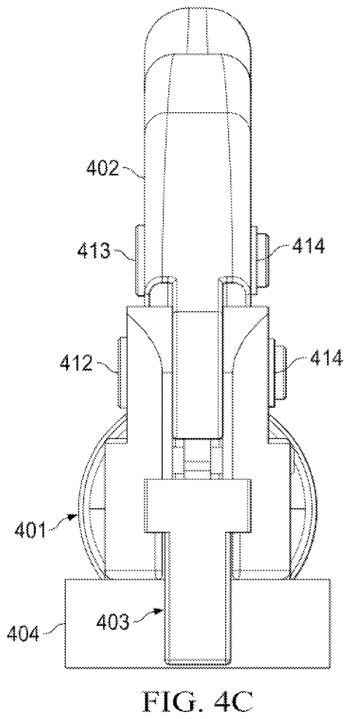

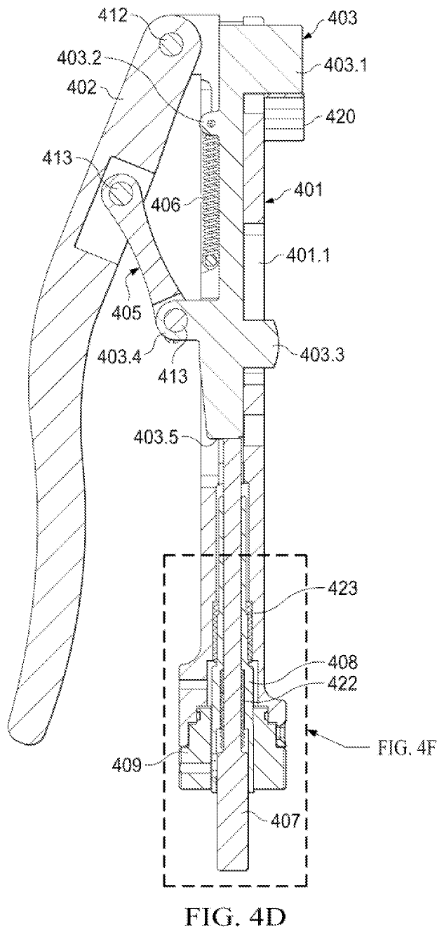

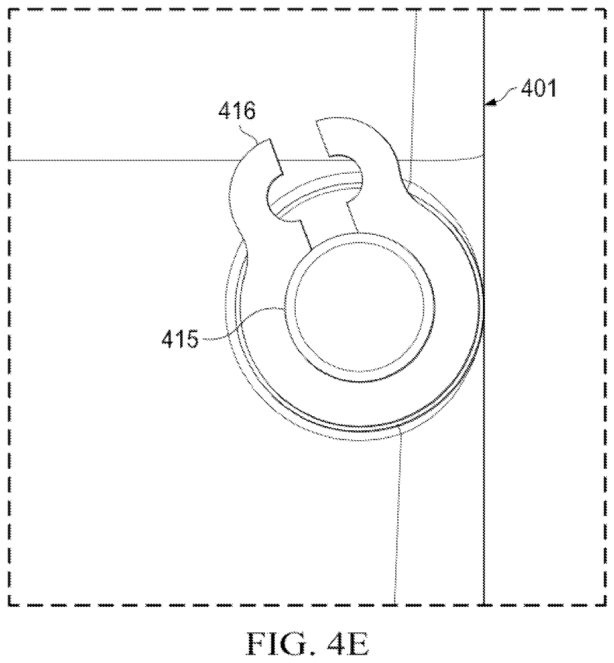

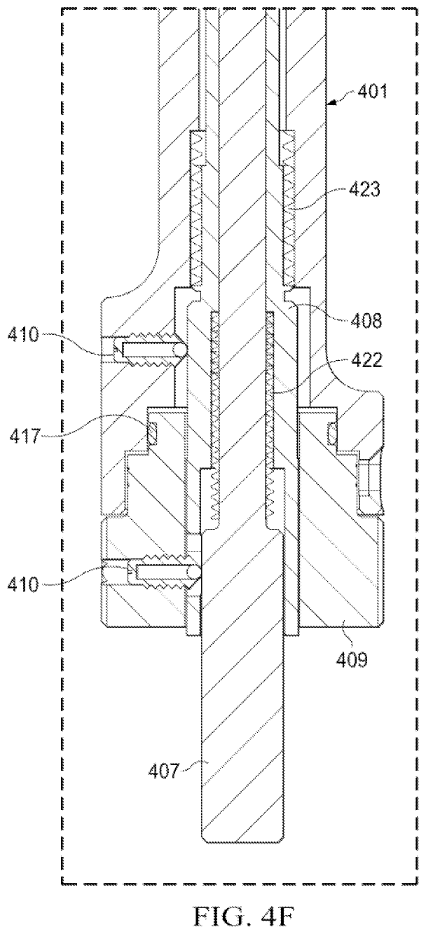

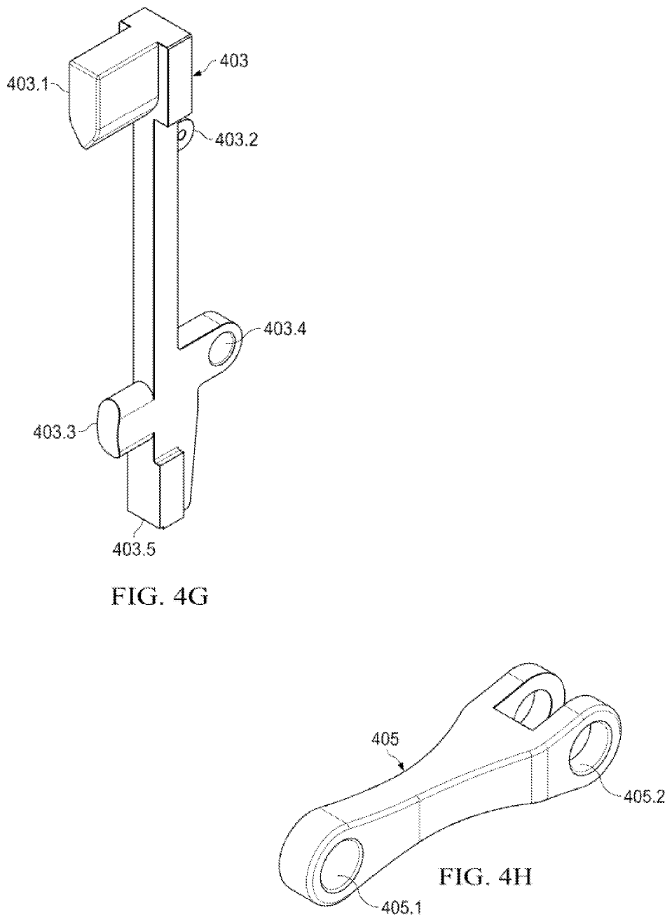

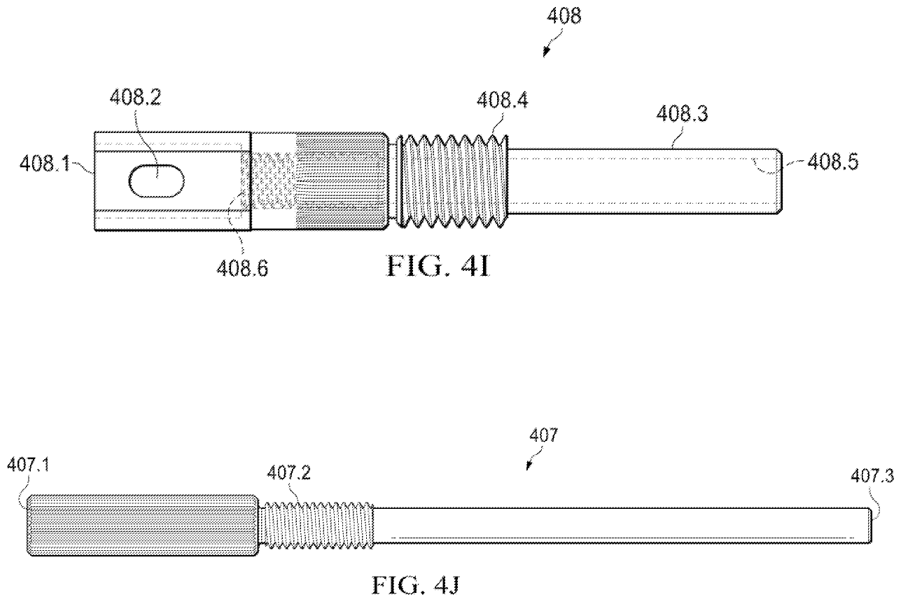

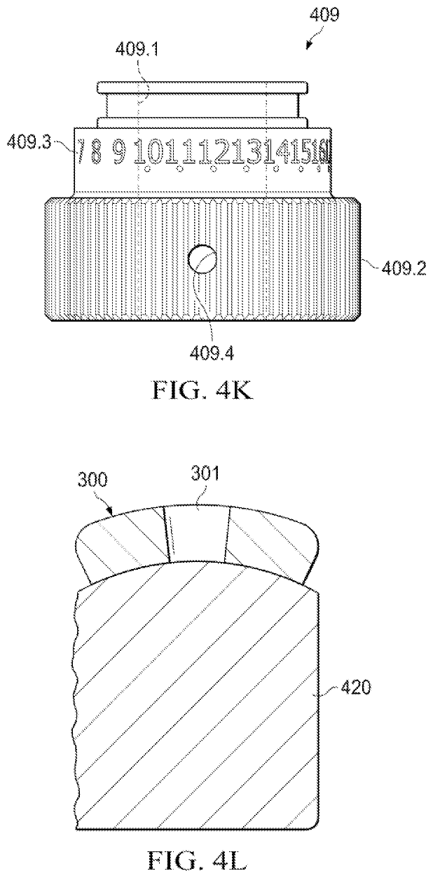

FIG. 4A-L shows several views of the bender 400, which is specially designed to allow accurate bending of, e.g., a toe plate 300 at bend line 314 from FIG. 3A. FIG. 4A shows a front view (handle to the back) with the toe plate 404 in place, ready to be bent. For simplicity it is shown as a simple rectangle, but it can be shaped as in FIG. 3 or as any other shape suitable for the joint fusion in question. FIG. 4B is a side view, 4C a top-down view, and 4D is a cross sectional view from the side. FIG. 4E is an enlargement of one of the pin hinges and retaining rings from FIG. 4B and FIG. 4F is an enlargement from 4D. FIG. 4G-K show various components alone and FIG. 4L shows a cross section of a post with convex upper surface that mirrors the cross section concave lower surface of the plate. For simplicity herein, the jaw end is considered the upper end of the bender, while the dial is the lower end.

In the side view of FIG. 4B, the handle 402 on frame body 401 is clearly visible. The main body 401 is hollow, providing a space for the travelling jaw 403 and angle set shaft 408 (408 not visible in this view, but see FIG. 4D or 4I) and calibrator and stop shaft 407 therein. In this figure the hollow cannot be seen, but it is cylindrical at the lower end and roughly of rectangular cross section at the upper end. However, it merely needs to be large enough and shaped to fit these components 403, 408, 407 and shape is otherwise not critical.

In FIG. 4B the frame body 401 is made as a unitary piece, but this is for prototyping purposes only, and we anticipate that posts 420 may be made as a separate snap in piece for the frame 401 so that the posts and back wall can be custom fitted to each toe plate, which will be available in at least 2-4 sizes. For example, the posts need to have convex top surface to fit the concave curve seen in FIG. 3C and the back wall may have two indents bracketing a concave bump to reflect the shape of bumps 321 and bi-concave section 322. Alternatively, just the posts are shaped to mirror the plate and a separate bender is sold for each plate.

As yet another alternative, small inexpensive plastic adaptors for each plate size and style can fit into a depression or receptacle or pin in the bender or on a protruding pin, allowing a large number of plates to all be bent using the same bender. Since the adaptors are mere plastic pieces, each having a front side that mirrors the various plates, and a back side that mirrors the single bender style, this may be a cost effective approach for a custom fit to each plate.

Shape mirroring is not essential, however, and a marking on the plate may be employed (e.g. a center line to line up with the upper pressure point, or three lines to line up with the three pressure points) instead of dedicated fitting components, allowing a variety of different plates to be bent using the same bender, provided their sizes are compatible with a given bender. With this methodology, e.g., all finger and toe plates may be bent with the same bender, but a larger joint, such as elbow, will probably need a larger bender.

In use, the handle 402 is lifted to provide a space between posts 420 and travelling jaw 403. Plate 404 is thus inserted into this space to fit on top of posts 420 and when correctly positioned, the travelling jaw 403 directly lines up with bend line 314 (from FIG. 3A). The dial 409 is adjusted (preferably pre-adjusted) as needed to move the angle set shaft 408 up or down—down allowing a greater degree of bend and up a lesser degree.

Spring 406 serves to tension the handle 402 so that it fits gently against plate 404, holding it steady until the user is ready to apply pressure. When the user applies a closing pressure to the handle 402, travelling jaw 403 is forced downwards as far as the calibrator and stop shaft 407 will allow and the plate 404 is thereby accurately bent to the designated angle.

The various additional parts including draglink 405 connecting handle 402 to main body 401, spring 406 tensioning the handle 402 and travelling jaw 403 via drag link 405, and the various couplings holding the parts movably together. Here we have used pin link 413, pin hinge 412, 415 (two sizes), retaining ring 414, 416 (two sizes), but any other fasteners can be used that allow the needed movement between the parts.

The details of the angle dialing components are shown in the cross section of FIG. 4F wherein the dial 409 is seen, along with calibrator and jaw stop 407 and angle set shaft 408. Templating view 410, in this case ball detents, are seen along with c-clamp 417, which serve to reversibly affix the parts. Angle set shaft 408 mates with dial 409 allowing the angle to be set by the user, and calibrator and jaw stop 407 providing both fine calibration and the stopping mechanism for the travelling jaw 403.

This embodiment of the device has two sets of threaded parts, including calibration threads 422 and dial threads 423. A single set of threads would suffice, but the dual system allows fine calibration of the dial. In more detail, the external threads of calibrator and jaw stop 407 mate with the internal threads of angle set shaft 408 to provide calibration threads 422 allowing fine calibration by turning the distal-most end of calibrator and jaw stop 407, which protrudes beyond dial 409. The external threads of 408 mate with the internal threads of hollow body 401 to form dial threads 423, wherein turning dial 409 moves the combined parts 407 and 408 up and down, thereby setting the bending angle by raising or lowering the stop 407. Some of these details may be better viewed in FIG. 4I-J.

FIG. 4G shows the travelling jaw 403 as a separate component. Here we see the subcomponents, such as bender point 403.1, spring connector 403.2, optional protrusion 403.3, and drag link connector 403.4. The bottom end of travelling jaw 403 merely provides stop surface 403.5 that abuts the top end of calibrator and stop shaft 407 when the travelling jaw is in its lowest position. Thus, the shapes at or near 403.5 are variable and need only fit inside the hollow and provide the requisite stopping function.

FIG. 4H shows the drag link 405 as a separate piece. Here, one end has a hole 405.1 and the other is forked and has a pair of holes 405.2 in each of the two prongs of the fork, but these shapes are dictated by the chosen fasteners and could be varied accordingly.

FIG. 4I shows the angle set shaft 408 in more detail, showing square end 408.1 connected to post 408.3. Hole 408.2 in square end 408.1 allows part 408 to be fixed to 407 once correctly calibrated, with e.g., a set screw or other fastener. Square end 408.1 fits into a square hole in dial 409, such that turning the dial 409 also turns part 408. The cylindrical portions 408.3 of 408 fit inside the hollow of main body 401, allowing rotation.

Just above the square section is an internally threaded section 408.6 which allows threaded coupling to 407. Above to 408.6 is an externally threaded section 408.4 which mates with a threaded area in the hollow inside body 401 (not visible, except in FIG. 4F) to formed dial threads 423 thereby allowing the height of the stop shaft to be set by dial 409. The entire body of angle set shaft 408 has a hollow center 408.5, allowing part 407 to be threaded therethrough. Hollow 408.5 is cylindrical so that cylindrical stop shaft 407 may rotate therein.

FIG. 4J shows the calibration and stop shaft 407 as a stand-alone part, showing ribbed end 407.1 which protrudes beyond dial 409. The friction from the optional ribs allows the user to turn the calibration and stop shaft calibration and stop shaft 407. Immediately proximal thereto are external threads 407.2 which mates with threads 408.6, and travelling jaw stop post is 407.3.

Another embodiment of a dial 409 is shown in FIG. 4K. The external surface of dial 409 is optionally ribbed 409.2, and there is a square hole 409.1 therein that mates with square end 408.1. Also seen is set screw hole with screw 409.4 which functions to allow calibration of the tool, plus degree markings 409.3.

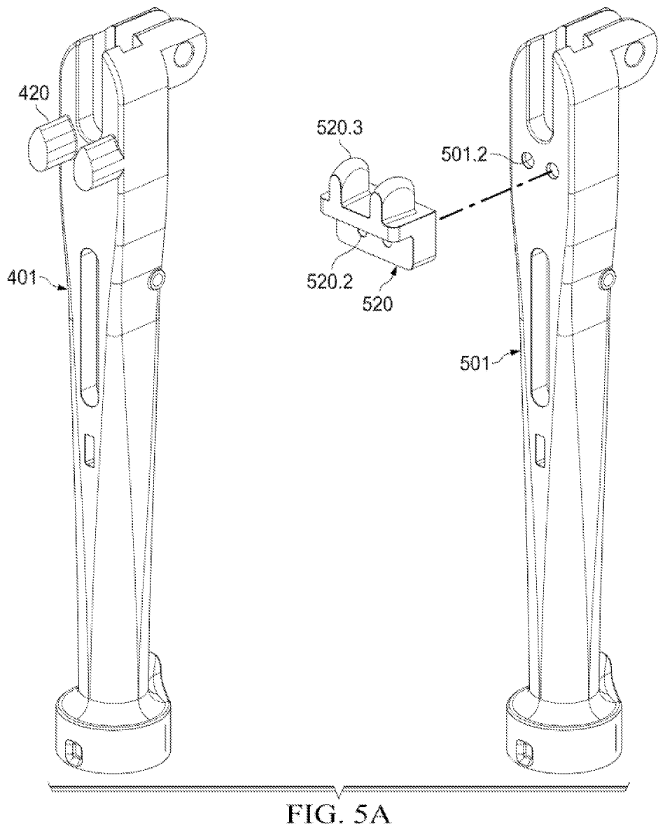

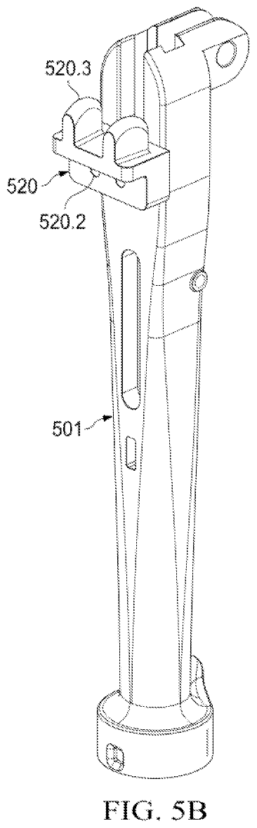

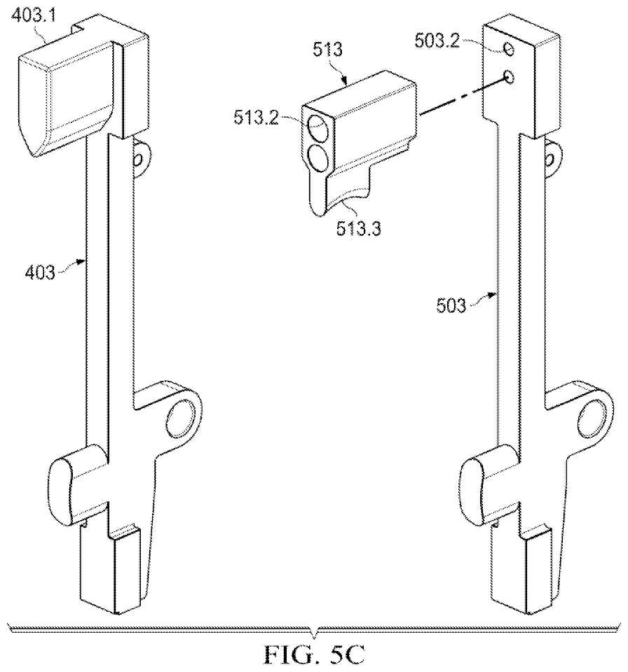

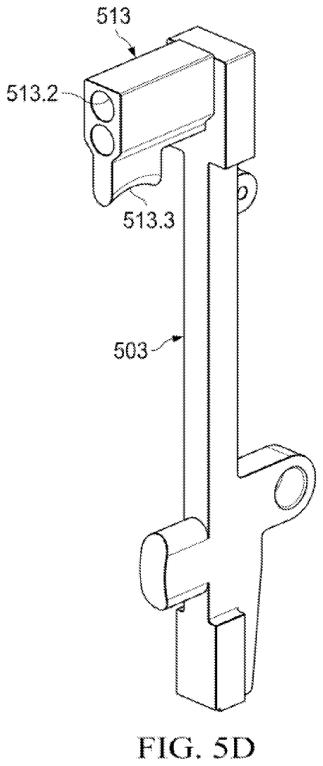

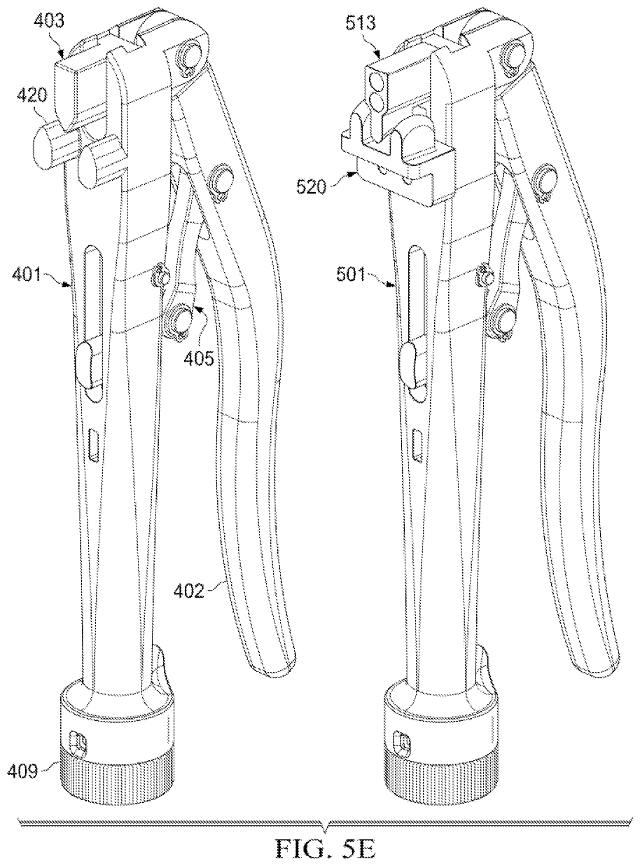

FIG. 5 shows an alternate embodiment with detachable posts and bender point, thus allowing customization of the tool for each type of plate. FIG. 5A-D show the differences in the new embodiment with that of FIG. 4 and like components not discussed again to avoid redundancy. FIG. 5E shows the original embodiment of FIG. 4 and new embodiment of FIG. 5 side by side.

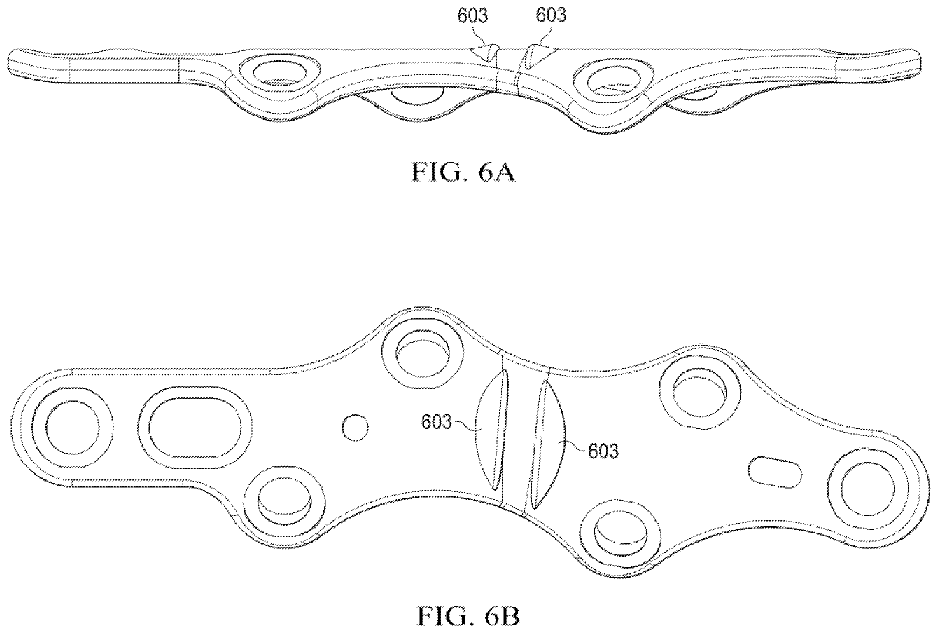

In more detail, FIG. 5A compares the original main body 401 with integral posts 420 and new main body 501 having no posts, but instead screw holes 501.2 therein. Detachable post component 520 has corresponding screw holes 520.2 and posts 520.3, here shown with a curved top surface to fit adjacent the bottom of the toe plate of FIG. 6. FIG. 5B shows the components assembled with screws. These components can also be snap fit or releasable pin fit, or any other easily removable but secure attachment means.

FIG. 5C compares the original travelling jaw 403 with bender point 403.1 and new travelling jaw 503 with screw holes 503.2 for attaching the separate bender point component 513 with corresponding screw holes 513.2. Here, the bender point 513.3 has a concave surface to fit adjacent the upper surface of the toe plate of FIG. 6. FIG. 5D shows 503 and 513 assembled.

FIGS. 6A and 6B shows an alternate embodiment of a toe plate with guide bumps 603, ensuring that the bender point sits in the exact position on the toe plate between guide bumps 603.

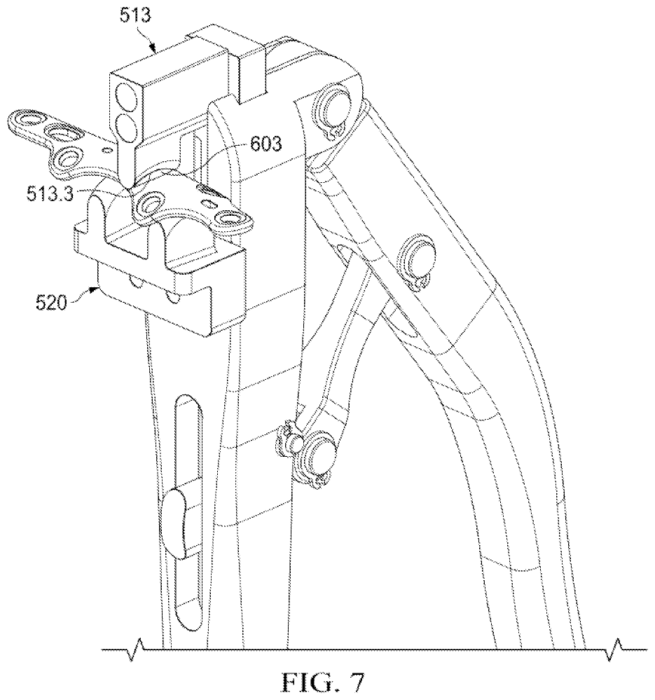

FIG. 7 shows the combined toe plate of FIG. 6 together with the alternate embodiment of the tool of FIG. 5 wherein the toe plate sits with guide bumps 603 bracketing the bender point 513.3.

### Preoperative Procedure

The patient typically presents to the clinic for evaluation of pain along the hallux metatarsophalangeal joint with or without deformities. The most common indications for consideration of a fusion are: 1) hallux rigidus—arthritis of the great toe metatarsophalangeal joint, 2) hallux valgus—bunion, and 3) hallux varus—inward turn of the great toe.

During the typical evaluation, 3 radiographic views of the foot are taken in the office—an anterior-to-posterior view, an oblique view, and a lateral view. These views are typically centered along the mid portion of the foot to have a broad assessment of the foot. If it is determined that a fusion of the hallux metatarsophalangeal joint is the best treatment option, a fourth view of the foot in the lateral plane is performed as follows:

The foot is placed on the foot board with the heel on the ¾ inch heel simulator and the patients stand with weight on that foot, the other foot pulled up out of the line of the beam. A small footstool can be placed forward of the X-rayed foot to make it easier for the patient to hold the foot out of the line of the beam. Care is taken to ensure that the toes are flat on the footboard and the radiographic beam is centered on the metatarsal head. This final radiograph—the “Templating View”—is used in order to allow for better measuring of the dorsal (top) aspect of the metatarsal shaft and the proximal phalanx.

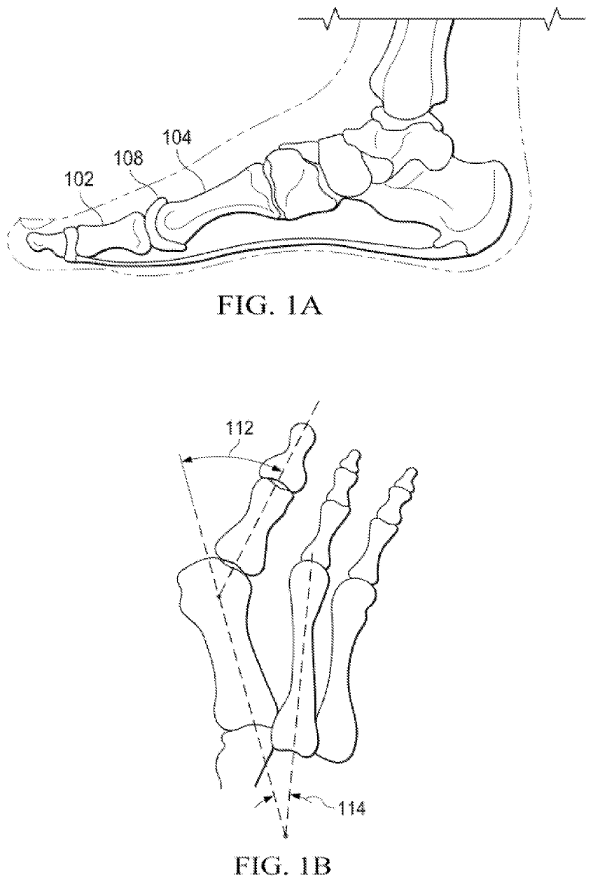

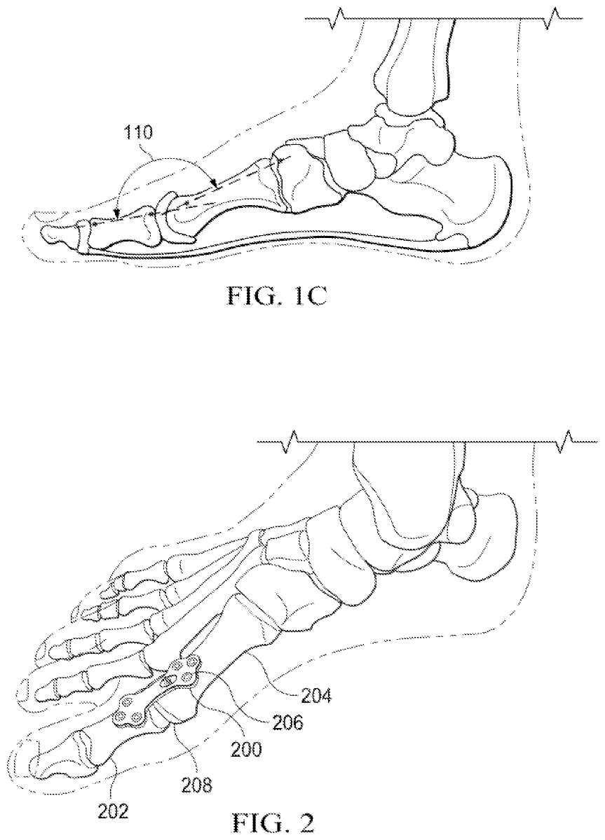

Once the templating view radiograph is available, a measurement of the angle between the constant portion of the metatarsal shaft dorsally and the constant portion of the proximal phalanx dorsally is performed as shown in FIG. 1C. Typically, the radiograph is digital and software computes the angle, but this is not essential, and the method can also be practiced with film. The surgeon or the AI will mark the beginning and end of the straight portions of the two bones and measure the angle created by intersection of the two lines created by joining the two points on a given bone.

### Intra-Operative Procedure

A standard dorsal—top of the foot—approach to the hallux metatarsophalangeal joint is carried out according to methods known in the art and described briefly here.

The dissection is carried down through the skin and the subcutaneous tissue with the scalpel. Scissors are then used to explore the proximal aspect of the incision for the medial branch of the superficial peroneal nerve. With the nerve protected, the extensor hallucis longus tendon is exposed.

The extensor hallucis longus tendon is then carefully elevated away from the underlying periosteum and capsule and retracted medially or laterally in order to expose the dorsal capsule of the hallux metatarsophalangeal joint.

The capsule is then incised along the mid portion following the same line of the incision and the tissues are elevated medially and laterally. It is important to release the medial and lateral capsular ligaments to allow for proper plantarflexion of the joint for preparation of the fusion surfaces. The toe is then plantarflexed—bent down—to allow for preparation of the metatarsal head. With the toe bent down, the hallux metatarsal is cannulated—pinned—with a K-wire following the line of the bone.

With the toe pinned, a cup reamer is then used to prepare the surface of the metatarsal head and remove the main layer of residual cartilage.

Once the main layer of cartilage is removed, the reamer and the pin are removed, and attention is then directed toward the base of the proximal phalanx. The phalanx is pinned with a K-wire of the same size following the axis of the bone. The base of the proximal phalanx is then reamed using a cone reamer over the above wire.

After the cartilage is removed, the reamer and the K-wire are removed from the joint. Both sides of the joint are then evaluated for any residual cartilage which, if present, is removed with a key elevator, curette, or rongeur. The wound is then copiously irrigated to remove any debris. The wound is dried with suction and both sides of the joint are drilled with a small drill bit to stimulate bleeding and aid in the fusion of the joint.

Traditionally, the next step in this procedure involves placing the toe in proper alignment and pinning the toe with a K-wire while holding that alignment. The position of the toe is then evaluated with a foot plate to make sure that it matches the posture of the other toes and allows the patient to use a heel of up to 1″. The problem with this approach is that it is user dependent, not reproducible, and is time consuming. To avoid this, we developed a plate first approach to the fixation.

### Plate-First Fixation

While the surgeon is performing the approach to the toe, the 0 (zero) degree plate is opened in sterile fashion and a sterile bender described herein to give the plate the proper degree of dorsiflexion based on the pre-operative template. Alternatively, this can be done in advance and the plate autoclaved or otherwise sterilized obviating the need for sterile technique.

Once the toe has been prepared for fusion using the above technique, the plate is brought into the field and positioned over the joint. Care is taken to hold the toe in proper rotation and longitudinal alignment (valgus). The plate is then pinned to the bone on both sides of the joint with threaded olive wires in order to hold the toe in alignment with the plate.

The position of the toe is confirmed visually in both the front and lateral planes. A ¾″ template, similar to or the same as the one used to measure the angles pre-operatively, is then used to confirm that the toe position matches the templated position.

After confirmation of proper alignment and position of the plate visually and radiographically, the trans-articular screw is addressed. While the plate is holding the toe in proper alignment, a K-wire from the cannulated screw set is brought into the field and the toe is pinned. Placement of the K-wire is confirmed with fluoroscopy. A small incision is then made at the entry site of the wire into the skin and the wire is drilled with a cannulated drill bit. The screw is then inserted over the wire and across the joint.

Prior to compressing the joint, the proximal olive wire is removed and only the wire in the oblong wire hole is left in place. This allows for compression of the joint while the plate is still over the joint, the plate can move proximally over the oblong hole without a change in overall position of the plate.

Once compressed, the only thing left to do is drill the holes on the plate and replace with screws. There is no need to reposition the plate in any way. When all of the hardware has been placed, surgery is completed and final radiographs are taken in the anteroposterior and lateral planes to confirm hardware placement and toe position.

Done in this manner, the procedure is reproducible in the vast majority of cases without having to depend on eyeballing of the position of the toe or the experience of the surgeon. It is also performed knowing that the position of the toe will allow for the patient to wear a standard heel as that measurement was performed with the patient weightbearing.

The present invention was exemplified in detail with respect to a hallux plate, bender and foot guide. However, this is exemplary only, and the invention can be broadly applied to other joints, such as e.g., other toes, fingers, elbows, and the like, and the components reshaped as needed for the different joints.

The following references are incorporated by reference in their entirety for all purposes.

U.S. Pat. No. 5,564,302 Orthopedic bone plate bending irons

U.S. Pat. No. 8,167,918 Orthopedic plate for use in the mtp joint

U.S. Pat. No. 8,419,745 Bone plate bender system

U.S. Pat. No. 9,615,874 Bone plate shaping system

The following commercial system are also available:

Synthes large plate bender

Arthrex MaxForce MTP compression plate

Acumed MP fusion plate

Biomet ALPS MP fusion plate

In2Bones Neo-fit MP fusion plates

Medartis Hallux System 2.8

OrthoSolutions Volition Foot Plating System

Synthes 2.4/2.7 Variable Angle Forefoot/Midfoot Set

Wright Medical Ortholoc MTP fusion plate

Wright Medical Ortholoc 2 Crosscheck MTP fusion plate.

1) A bender for precise bending of a separate joint fusion plate, said bender comprising:
a) a main body having a hollow therein and having first and second pressure points protruding from an exterior surface near a top end of said main body and a first slot between said first and second pressure points;
b) a stop shaft inside said hollow and means for controlling a height of said stop shaft inside said hollow;
c) a travelling jaw having a lower end at least partially inside said hollow, said travelling jaw having a third pressure point at an upper end of said travelling jaw, said third pressure point positioned between said first and second pressure points in said slot;
d) said travelling jaw able to travel up to allow placement of a separate joint fusion plate between said first and second pressure points and said third pressure point, and able to travel down to meet said stop shaft;
e) a handle operatively connected to said main body and to said travelling jaw, said handle for raising and lowering said travelling jaw and for applying a pressure to said third pressure point when said separate joint fusion plate is positioned in a stable and unmoving position supported by said first and second pressure points, thereby bending said separate joint fusion plate.

2) The bender of claim 1, wherein said means for controlling said height of said stop shaft is a rotating dial at a bottom of said main body that raises and lowers said stop shaft via a threaded region that mates with a threaded region inside said hollow.

3) The bender of claim 2, wherein said dial has markings thereon for indicating a degree of bending.

4) The bender of claim 1, wherein said travelling jaw is biased to return to a lowered position.

5) The bender of claim 1, wherein said travelling jaw is spring biased to return to a lowered position with a spring connected to said main body and to said travelling jaw.

6) The bender of claim 1, wherein said handle is hingedly connected to said main body and to said travelling jaw via a drag link, said main body having a second slot opposite said first slot, said second slot allowing said drag link to travel up and down said main body with said handle.

7) The bender of claim 1, wherein said handle is hingedly connected to said main body and to said travelling jaw via a drag link, said main body having a second slot opposite said first slot, said second slot allowing said travelling jaw to travel up and down with said handle, wherein said travelling jaw is spring biased to return to a lowered position, said spring connected to said main body and to said travelling jaw.

8) The bender of claim 1, wherein said main body, said handle, and said travelling jaw are made of metal.

9) The bender of claim 1, wherein said first and second pressure points are integral to said main body and wherein said third pressure point is integral to said travelling jaw.

10) The bender of claim 1, wherein said first and second pressure points are not integral to said main body, but comprise a first separate attachable component, and wherein said third pressure point is not integral to said travelling jaw but comprises a second separate attachable component.

11) The bender of claim 1, wherein said first and second pressure points have an upper surface that mirrors a lower surface of said separate joint fusion plate and said third pressure point has a lower surface that mirrors a upper surface of said separate joint fusion plate.

12) A kit for joint fusions, said kit being a container comprising:
a) the bender of claim 1; and
b) a plurality of metal joint fusion plates having a shape, said shape designed to i) fit against two bones to be fused and a plurality of holes for attachment means to travel therethrough and into said two bones and ii) fit into said bender.

13) The kit of claim 12, further comprising one or more of:
a) instructions for use;
b) at least two known angle bent plates for calibrating said bender;
c) said plurality of metal joint fusion plates being in a plurality of sizes; or
d) said shape fitting against a phalanx bone and a metatarsal bone.

14) A method for fusing two bones, said method comprising:
a) obtaining a radiograph of two bones to be fused, said two bones positioned to have a desired angle therebetween;
b) measuring said desired angle from said radiograph;
c) bending a joint fusion plate to have said desired angle using the bender of claim 1; and
d) surgically installing said bent joint fusion plate onto said two bones.

15) The method of claim 14, wherein said bent joint fusion plate is sterilized after bending and before use.

16) The method of claim 14, wherein said bent joint fusion plate is sterilized before bending and said bending step c) is performed in a sterile manner.

17) The method of claim 14, said method being for fusing a phalanx bone and a metatarsal bone, said method comprising:
a) obtaining a radiographic image of a foot having a phalanx bone and a metatarsal bone to be fused by positioning said foot on a ¾-1 inch heel block, applying weight to said foot and taking said radiographic image;
b) measuring an angle between said phalanx bone and said metatarsal bone from said radiographic image;
c) bending a joint fusion plate to have said angle using the bender of claim 1; and
d) surgically installing said bent joint fusion plate onto said phalanx bone and said metatarsal bone.

18) The method of claim 17, wherein steps a-c) occur before step d) is initiated.

19) The method of claim 17, wherein steps a-b) occur before step d) is initiated.
